(19) United States
(12) Patent Application Publication
(10) Pub. No.: US 2026/0241194 A1
(43) Pub. Date: Aug. 20, 2026

(54) METHOD FOR IMPROVING RETINAL VASCULAR CIRCULATION USING RADIO FREQUENCY (RF) MICROWAVES, RF EMITTING CIRCUIT USING THE SAME AND USE THEREOF

(71) Applicant: An-Chih CHANG, Tainan City (TW)

(72) Inventor: An-Chih CHANG, Tainan City (TW)

(21) Appl. No.: 19/543,091

(22) Filed: Feb. 18, 2026

(30) Foreign Application Priority Data
Feb. 20, 2025 (TW) 114106341

(51) Int. Cl. A61N 5/02; A61N 5/04

(52) U.S. Cl. CPC A61N 5/022; A61N 5/04

(57) ABSTRACT

A method for improving retinal vascular circulation using radio frequency (RF) microwaves, an RF emitting circuit using the same and use thereof are provided to apply a non-thermal effect to an eye by emitting the RF microwaves with a frequency between 30 MHz and 3 GHz, thereby substantially imperceptibly regulating retinal vessel density (VD). That is, it can increase the too low vessel density and reduce the too high vessel density, thereby optimizing the blood flow distribution of retinal capillary to improve vascular circulation.

### CROSS-REFERENCE TO RELATED APPLICATION

This non-provisional application claims priority under 35 U.S.C. § 119(a) to Patent Application No. 114106341 filed in Taiwan, R.O.C. on Feb. 20, 2025, the entire contents of which are hereby incorporated by reference.

### BACKGROUND

### Technical Field

The present invention relates to bioelectromagnetic technology, and in particular to a method for improving retinal vascular circulation using radio frequency (RF) microwaves, an RF emitting circuit using the same and use thereof.

### Related Art

With the advancement of Radio Frequency (RF) technology, electronic devices utilizing RF technology (hereinafter referred to as RF devices) have grown rapidly in human body-related applications, such as medical care, aesthetics, health care and personal care.

For non-medical applications (such as aesthetics, home health care or home care), energy-based RF devices act on the human body or the skin through electrodes in a non-invasive or invasive manner. A non-invasive RF device uses electrodes attached onto the skin to generate a thermal effect from RF current in skin tissue, thereby triggering reversible denaturation of collagen in the dermis, stimulating the formation of new collagen in a healing process, and further achieving a lifting and firming effect on the skin. An invasive RF device uses a positive electrode tip to touch a treated area, and use a negative electrode which is a conductive negative electrode plate to touch another part of the human body. The invasive RF device applies RF energy through the positive and negative electrodes, and the RF energy may be radiated from the epidermis downward into the subcutaneous layer. Therefore, the RF energy achieves deeper penetration, which helps enhance the skin tightening effect.

### SUMMARY

However, when a radio frequency (RF) device is used by directly touching the skin or a treated area with metal electrodes, RF current directly acts on the human body, stabbing pain and uncomfortable feeling may easily occur, and too high local skin temperature may be easily caused, thereby posing a risk of thermal injury. In addition, an existing RF device cannot be used for improving the retinal vascular circulation. This is because the eyeball has a delicate and fragile structure, and it cannot withstand a high temperature. Particularly, the retina, located at the back of the eye, is extremely sensitive to a temperature change. Too high temperature may cause irreversible injury. Therefore, up to now, there is no effective and safe device or method available to directly improve the retinal vascular circulation. However, with long-time use of 3C products, the workload and blood supply demands of the retina may be greatly increased. Therefore, whether the retinal vascular circulation may be improved or not through a non-thermal effect has become a major topic in vision maintenance.

In view of this, the present invention provides a method for improving retinal vascular circulation by regulating retinal vessel density (VD) through RF microwaves, an RF emitting circuit using the method and use thereof. A non-thermal effect may be applied to an eye by emitting the RF microwaves with a frequency between 30 MHz and 3 GHz, so that the problems such as stabbing pain, uncomfortable feeling and/or too high local temperature when RF device directly acts on the human body may be avoided, while achieving regulating the retinal VD density to improve the vascular circulation.

In some embodiments, a method for improving vascular circulation using RF microwaves to regulate the retinal VD includes: positioning an RF emitting circuit within a range of 3 cm to 10 cm away from an eye; driving the RF emitting circuit to emit an RF microwave with a frequency between 30 MHz and 3 GHz; and applying a non-thermal effect to the retinal area of the eye by emitting the RF microwave.

In some embodiments, the RF emitting circuit is a passive RF circuit.

In some embodiments, the RF emitting circuit is an active RF circuit.

In some embodiments, the RF emitting circuit is a glasses clip or a pair of glasses.

In some embodiments, use of an RF emitting circuit for regulating retinal VD to improve vascular circulation is provided, where the RF emitting circuit is configured to generate an RF microwave with a frequency between 30 MHz and 3 GHz.

In some embodiments, an RF emitting circuit for regulating retinal VD includes an emitting module and a control module. The control module is connected to the emitting module, and is configured to control the emitting module to generate an RF microwave with a frequency between 30 MHz and 3 GHz.

In some embodiments, the control module is a passive chip.

In some embodiments, the RF emitting circuit further includes a power supply module. The power supply module is connected to the control module, and is configured to supply power to the control module.

In some embodiments, the RF emitting circuit further includes a pair of glasses, and the emitting module and the control module are disposed on the pair of glasses.

Based on the above, the method for improving retinal vascular circulation using RF microwaves, the RF emitting circuit using the same and the use thereof according to any of embodiments may be used for applying a non-thermal effect to an eye by emitting the RF microwaves with a frequency between 30 MHz and 3 GHz, thereby substantially imperceptibly regulating retinal VD and improving the vascular circulation.

### BRIEF DESCRIPTION OF THE DRAWINGS

FIG. 1 is a function block diagram of a radio frequency (RF) emitting circuit according to an embodiment.

FIG. 2 is a schematic outside view of an example of the RF emitting circuit in FIG. 1.

FIG. 3 is an exploded view of the RF emitting circuit in FIG. 2.

FIG. 4 is a schematic outside view of another example of the RF emitting circuit in FIG. 1.

FIG. 5 is a schematic circuit diagram of an example of the RF emitting circuit in FIG. 1.

FIG. 6 is a function block diagram of an RF emitting circuit according to another embodiment.

FIG. 7 shows a detection result of radiated emission of the RF emitting circuit in FIG. 5.

FIG. 8 is a schematic diagram of an example of retinal angiography image.

FIG. 9A to FIG. 9D are bar charts showing experimental data of VD value changes.

FIG. 10A and FIG. 10B are line graphs showing experimental data of total VD value changes over time.

FIG. 11A and FIG. 11B are line graphs showing experimental data of VD value changes over time in low-density areas.

FIG. 12A and FIG. 12B are line graphs showing experimental data of VD value changes over time in medium-density areas.

FIG. 13A and FIG. 13B are line graphs showing experimental data of VD value changes over time in high-density areas.

FIG. 14A and FIG. 14B are bar charts showing experimental data on the effect of disease on VD value changes.

FIG. 15A and FIG. 15B are distribution diagrams showing experimental data on response rate analysis.

### DETAILED DESCRIPTION

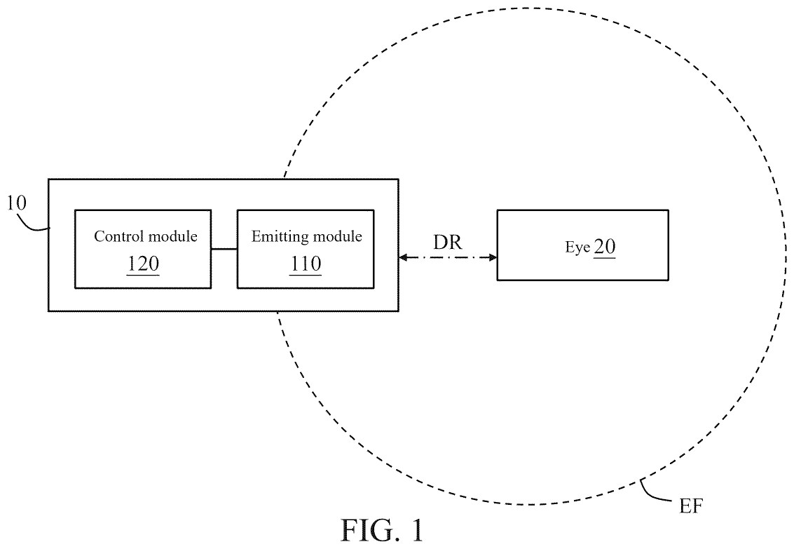

Referring to FIG. 1, a radio frequency (RF) emitting circuit 10 is configured to generate an RF microwave EF with a non-thermal effect. Specifically, the RF emitting circuit 10 may be disposed around a target eye 20 in a non-contact manner, and may generate an electromagnetic field (i.e., RF microwave EF) that exposes the eye 20 to the generated electromagnetic field. This non-contact electromagnetic field is thereby utilized to induce a non-thermal effect in a retinal area of the eye 20. A frequency of the RF microwave EF is between 30 MHz and 3 GHz.

In some embodiments, referring to FIG. 1, the RF emitting circuit 10 includes an emitting module 110 and a control module 120. The control module 120 is connected to the emitting module 110, and is configured to control the emitting module 110 to generate an RF microwave EF with a frequency between 30 MHz and 3 GHz and a signal strength between 10 dB V and 30 dBμV. In other words, the frequency of the RF microwave EF falls within a bandwidth range of an ultra high frequency (UHF).

In some embodiments, the frequency of the RF microwave EF is between 30 MHz and 1000 MHz. In some embodiments, the frequency of the RF microwave EF may be between 600 MHz and 1000 MHz. In some embodiments, the frequency of the RF microwave EF may be 900 MHz.

In some embodiments, an operating distance DR of the RF emitting circuit 10 is between 3 cm and 10 cm.

In some embodiments, in use, the RF emitting circuit 10 is first positioned within a range of 3 cm to 10 cm away from an eye 20, then, the RF emitting circuit 10 is driven to emit an RF microwave EF so as to apply a non-thermal effect to the retinal area of the eye 20 using the emitted RF microwave EF. In some embodiments, for a retinal area with too low vessel density, the RF microwave EF emitted by the RF emitting circuit 10 may promote the vessel generation in the retinal area through the non-thermal effect to improve its vessel density. For a retinal area with too high vessel density, the RF microwave EF emitted by the RF emitting circuit 10 may decrease the vessel generation in the retinal area through the non-thermal effect to improve its vessel density.

In some embodiments, the RF emitting circuit 10 can regulate and control the VD of the retinal area of the eye 20 when applying the RF microwave EF with the non-thermal effect (even when the eye 20 is exposed to an electromagnetic field generated by the RF emitting circuit 10) to the eye 20. Therefore, this RF emitting circuit 10 is applicable to preparing an circuit for regulating and controlling retinal VD (such as a wireless RF circuit or a wearable circuit). In some embodiments, the vessels may be capillaries.

Specifically, the retinal VD refers to a dense degree of a vascular network in the retina. Both insufficient retinal VD and excessive new vessels may cause adverse effect on the eye's health. Therefore, the RF emitting circuit 10 may be used for non-medical purpose eye health care so as to improve the VD of a low VD area under the condition without the abnormal increase of capillary density in a normal area for improving the vascular circulation. According to the FDA Instrument No. 1010032059 issued by the Food and Drug Administration of the Department of Health of the Executive Yuan of the Republic of China, “improving vascular circulation” is not regarded as a medical efficacy.

In some embodiments, when the VD is excessively reduced, a non-flow area may be formed on the retina, so that the blood supply to the retina may be reduced, causing hypoxia. Long-term hypoxia may injure retinal cells, especially photoreceptor cells, causing diminution of vision. In some cases, too low VD is correlated to various eye diseases such as glaucoma, diabetic retinopathy subtypes, age-related maculopathy, or severe myopia. Therefore, in some embodiments, the RF emitting circuit 10 may also be used for relieving or avoiding the non-flow area so as to treat or prevent various eye diseases such as glaucoma, diabetic retinopathy subtypes, age-related maculopathy, or severe myopia.

In addition, patients without eye diseases may experience low retinal VD due to changes in the internal environment caused by their diseases. For example, diabetic patient's too high blood sugar may cause damage to the vascular endothelium and long-term damage to the microvascular system. For another example, cardiovascular diseases or chronic obstructive pulmonary diseases may cause chronic ischemia, hypoxia, and/or hypercapnia, consequently exacerbating the atrophy and lesions of retinal vessels. Therefore, in some embodiments, the RF emitting circuit 10 may also be used for reducing the damage of the stressful environment on the vascular endothelial cells.

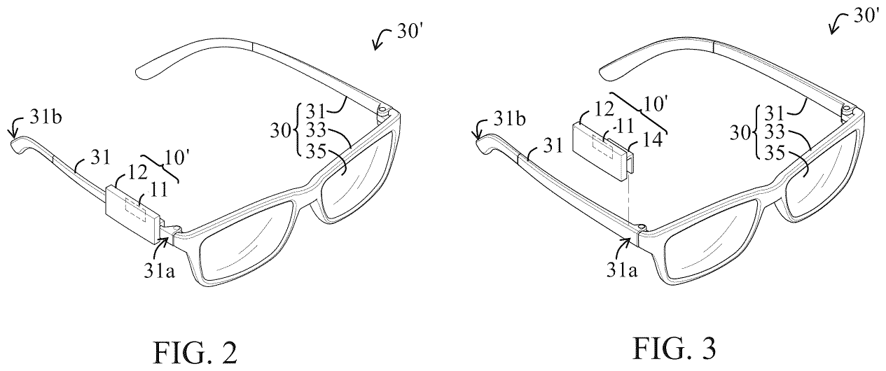

In some embodiments, the RF emitting circuit 10 may be an independent wireless RF circuit 10′, as shown in FIG. 2. Specifically, referring to FIG. 2, the wireless RF circuit 10′ includes an RF circuit 11 and a housing 12. The RF emitting circuit 11 is positioned in the housing 12, and is disposed in an accommodating space inside the housing 12. Therefore, the RF emitting circuit 11 is configured to emit an RF microwave EF with a frequency between 30 MHz and 3 GHz. When the wireless RF circuit 10′ is used, the housing 12 may be fixed onto a wearable circuit 30 in a fixing manner such as sticking, locking, buckling, or clamping. Based on this, a user may assemble the wireless RF circuit 10′ onto the wearable circuit 30 when necessary, so that the use of the RF emitting circuit 10 is more convenient. In an example, referring to FIG. 2 and FIG. 3, by taking the wearable circuit 30 being a pair of glasses as an example, the RF emitting circuit 10 may be a glasses clip (that is, the wireless RF circuit 10′ is a glasses clip). Therefore, the RF circuit 11 is disposed in the housing 12 of the wireless RF circuit 10′, and a clamping element 14 is disposed on an outer surface of the housing 12. In use, a glasses leg 31 of the pair of glasses is clamped between the housing 12 and the clamping element 14, so that the wireless RF circuit 10′ is fixed onto the pair of glasses. A structural design of the clamping element 14 is well known in the art, and will not be described in further detail herein.

In some other embodiments, the RF emitting circuit 10 may also be a wearable circuit 30′. That is, one of components of the wearable circuit 30′ is an RF circuit 11 with the above functions, as shown in FIG. 4. For example, by taking the wearable circuit 30′ being a pair of glasses as an example, the wearable circuit 30′ is provided with an RF circuit 11 embedded inside the pair of glasses (for example, a glasses frame or glasses lens 35), and in addition, the RF circuit 11 may emit an RF microwave EF with a frequency between 30 MHz and 3 GHz. Specifically, the RF circuit 11 may be embedded in the glasses leg 31 (as shown in FIG. 4) of the glasses frame or embedded in the glasses lens frame 33 or the glasses lens 35 (not shown) of the glasses frame.

In some embodiments, the glasses leg 31 is provided with a connecting end 31a and an ear hook end 31b which are opposite to each other. The glasses leg 31 is coupled to a glasses lens frame 33 (as shown in FIG. 2 and FIG. 3) or the glasses lens 35 (not shown) via the connecting end 31a. Therefore, the RF circuit 11 configured to emit the RF microwave EF may be disposed on the connecting end 31a of the glasses leg 31. For example, the wireless RF circuit 10′ used as the RF emitting circuit 10 is removably assembled onto the connecting end 31a of the glasses leg 31 (as shown in FIG. 2 and FIG. 3), or the RF circuit 11 is directly embedded in the connecting end 31a of the glasses leg 31 (as shown in FIG. 4).

In some embodiments, besides the above glasses, the wearable circuit 30/30′ may also be a head-worn device, such as a hair accessory, a cap, or a mask, and it is not limited in the present invention.

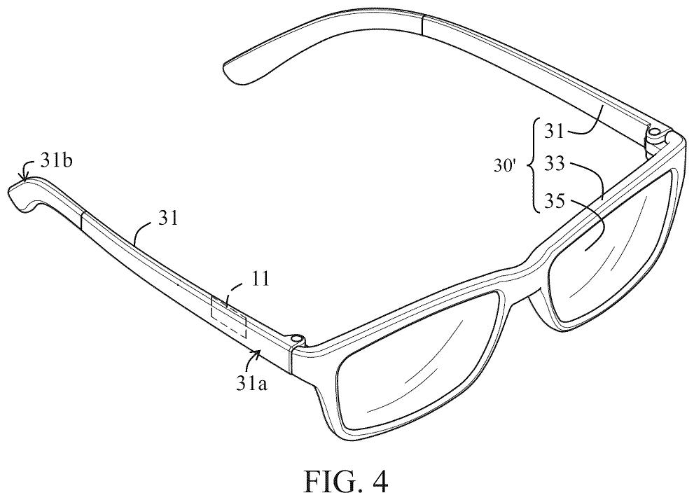

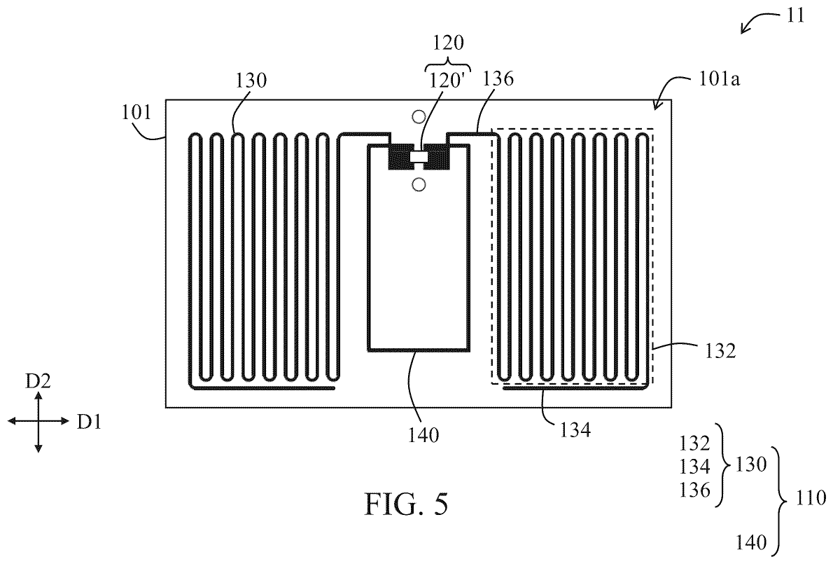

In some embodiments, the RF circuit 11 may be implemented with a circuit board 101, as shown in FIG. 5. In other words, referring to FIG. 1 and FIG. 5, the RF circuit 11 includes a circuit board 101, an emitting module 110 formed on a surface 101a of the circuit board 101, and a control module 120 disposed on the circuit board 101. The circuit board 101 may be a single-layer board or a multilayer board.

In some embodiments, the emitting module 110 may consist of an antenna pattern 130 and a matching pattern 140 (as shown in FIG. 5), but it is not limited thereto. In practical application, the emitting module 110 may also only consist of an antenna pattern 130, or may consist of an antenna pattern 130 and one or more signal processing components such as a matching pattern 140, an amplifier or a filter. Referring to FIG. 5, the matching pattern 140 and the antenna pattern 130 may be circuit traces on any one surface 101a of the circuit board 101.

In some embodiments, the control module 120 may be an RF chip 120′, as shown in FIG. 5. The RF chip 120′ is electrically connected to the circuit trace of the matching pattern 140 and is fixed (or welded) to an electronic component on the circuit board 101.

In some embodiments, two antenna patterns 130 are provided, and the two antenna patterns 130 are symmetrical to each other. Therefore, the two antenna patterns 130 have similar back-and-forth bending structures 132. The back-and-forth bending structure 132 of each antenna pattern 130 is provided with a plurality of vertical traces disposed and arranged in a first direction D1 and extending in a second direction D2. At least more than 10 (for example, 15) vertical traces of each antenna pattern 130 are provided. The RF chip 120′ is couped to one side edge (referred to as a first side edge hereafter) of the matching pattern 140. A first end of the back-and-forth bending structure 132 of each antenna pattern 130 is coupled to the first side edge of the matching pattern 140 through a first connecting trace 136. Each antenna pattern 130 is further provided with a horizontal trace 134 extending in the first direction D1. A first end of the horizontal trace 134 of each antenna pattern 130 is coupled to a second end of the back-and-forth bending structure 132 thereof, and a second end of the horizontal trace 134 is a free end located closer to the matching pattern 140 relative to the free end. The first end and the second end of the back-and-forth bending structure 132 of each antenna pattern 130 are positioned at two diagonal corners of the back-and-forth bending structure 132.

In some other embodiments, the RF emitting circuit 10 may be implemented with a combination of a circuit board and a metal sheet (not shown). Specifically, the matching pattern 140 may be a circuit trace on any one surface 101a of the circuit board 101. The RF chip 120′ is an electronic component fixed (or welded) onto the circuit trace of the circuit board 101, thereby being electrically connected to the matching pattern 140. The antenna pattern 130 is formed from a metal sheet, and in addition, a signal feed point on the metal sheet is directly welded onto or connected through a conductor onto the circuit trace on any one surface 101a of the circuit board 101, thereby being electrically connected to the matching pattern 140.

In some embodiments, the RF emitting circuit 10 may be a passive circuit. That is, the RF circuit 11 is a passive RF circuit. That is, the RF chip 120′ is a passive chip. In this case, the RF chip 120′ may be activated by sensing the ambient electromagnetic field via the antenna pattern 130. That is, the RF emitting circuit 10 may sense and respond to the ambient electromagnetic field to be activated and may emit the RF microwave EF through the antenna pattern 130, so as to apply a non-thermal effect to the retinal area of the eye 20 for regulating the retinal VD of the eye 20. In some embodiments, according to a CISPR 11 Class B measurement method, a signal strength of the RF microwave EF emitted by the passive RF circuit (i.e., the electric field strength of the electromagnetic field) may be between 10 dBμV/m and 30 dBμV/m.

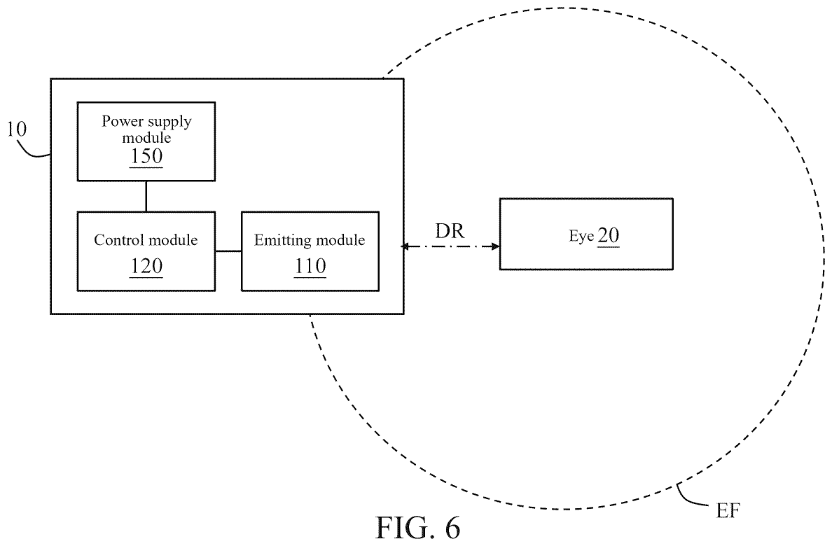

In some other embodiments, the RF emitting circuit 10 may be an active circuit. That is, the RF circuit 11 is an active RF circuit. That is, the RF chip 120′ is an active chip. In this case, referring to FIG. 6, the RF emitting circuit 10 may further include a power supply module 150. In addition, the power supply module 150 is connected to a control module 120 (for example, to the RF chip 120′). In use, the power supply connection delivers electric power to the control module 120, so that the control module 120 controls the emitting module 110 to emit the RF microwave EF, so as to apply a non-thermal effect to the retinal area of the eye 20 for regulating the retinal VD of the eye 20. In some embodiments, the signal power of the active RF circuit is between 0 dBm (decibel milliwatt) and 30 dBm.

### Example 1: Human Trial

### A. Subject Conditions

The inclusion criteria for the subjects are as follows: no gender restriction, aged 40 to 85 years, required to wear glasses for at least 8 hours every day, and self-reported recent vision decline and/or eye fatigue. Furthermore, the subjects also need to be able to undergo fundus microangiography (optical coherence tomography angiography, OCTA), with clear and distinguishable images.

The exclusion criteria for the subjects are as follows: having undergone ophthalmic surgery for diabetic retinopathy, intraocular pressure exceeding 21 mmHg, failure to be tested according to the trial schedule, inability to undergo fundus microangiography or poor image quality, and development of new eye diseases and/or receipt of new medication during the trial.

### B. Design of RF Emitting Circuit 10

The glasses clip in experimental group is the wireless RF circuit 10′ equipped with the RF circuit 11 shown in FIG. 5, as shown in FIG. 2 and FIG. 3. The glasses clip in control group is only provided with an outer casing (i.e., the housing 12 and the clamping element 14), but is provided with no RF circuit 11.

The RF chip 120′ of the RF circuit 11 uses a passive UHF RF emitter (IMPINJ MONZA R6) having an operation frequency set to 900 MHz and an output signal strength set to 20 dBμV. During the test, the RF chip 120′ emits the RF microwaves EF through the antenna pattern 130 at a frequency of once per second, with a duration of 0.1 seconds per emission.

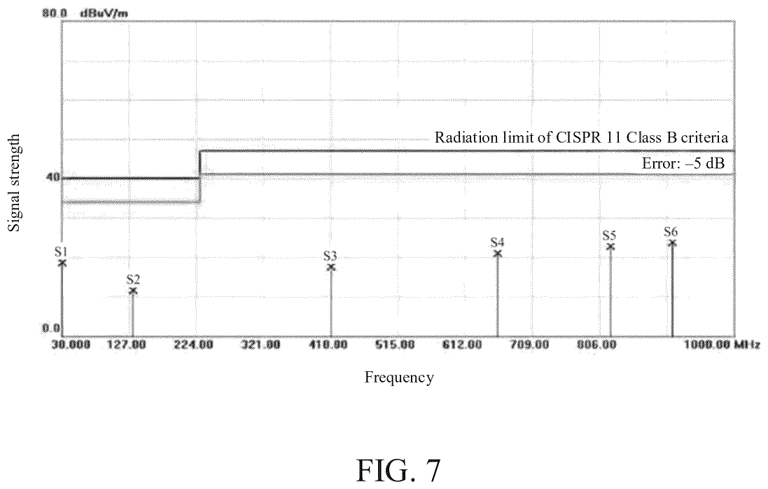

According to the CISPR11 Class B test criteria, a QP (Quasi-Peak) detection mode is adopted to perform radiated emission detection on the glasses clip in experimental group. Measurement results are presented in Table 1 and FIG. 7. Compliance with the CISPR11 Class B test criteria indicates that a tested product qualifies as an industrial, scientific, or medical RF device suitable for residential use. From Table 1 and FIG. 7, it can be seen that the RF microwaves EF emitted by the RF circuit 11 generated pulses S1 to S6 in 6 frequency points, and the electric field intensities of these pulses S1 to S6 did not exceed the limit. Therefore, an emitting frequency range of the RF microwave EF of the RF circuit 11 is between 30 MHz and 1000 MHz.

TABLE 1

| | | Reading | | | | |
|---|---|---|---|---|---|---|
| | Freq. | Level | CF | Measurement | Limit | Over |
| | (MHz) | (dBμV) | (dB/m) | (dBμV/m) | (dBμV/m) | (dB) |
| S1 | 30.00 | 20.80 | −2.30 | 18.50 | 40.00 | −21.50 |
| S2 | 133.24 | 20.50 | −8.99 | 11.51 | 40.00 | −28.49 |
| S3 | 418.32 | 20.90 | −3.34 | 17.56 | 47.00 | −29.44 |
| S4 | 659.09 | 21.70 | −0.83 | 20.87 | 47.00 | −26.13 |
| S5 | 822.77 | 21.70 | 1.07 | 22.77 | 47.00 | −24.23 |
| S6 | 911.07 | 21.60 | 2.09 | 23.69 | 47.00 | −23.31 |

In Table 1, the field “Freq.” represents the frequency point; the field “Reading Level” represents a value read from a detection instrument; the field “CF” represents a correct factor; the field “Measurement” represents a confirmed final value of each frequency point; the field “Limit” represents a limit value, i.e., the Measurement shall not exceed this Limit; and the field “Over” represents the degree of exceeding the Limit, i.e., a calculation result obtained by subtracting the Limit from the Measurement. A calculation formula of the Measurement is as follows: Measurement=Reading Level+Correct Factor. CF=AF+CL−AG. In the formula, AF represents an antenna corrector factor, CL represents a coaxial signal cable attenuation loss, and AG represents a signal amplifier gain. A negative number for Over indicates that the limit value has not been exceeded, which means it meets the requirements of the test criteria.

### C. Experiment Design

This trial is conducted in a double-blind manner, meaning that neither the subjects nor the optometrists responsible for the examination know whether the glasses clips clamped on the glasses worn by the subjects have RF functions or not.

Therefore, 32 subjects meeting the inclusion criteria first underwent retinal microangiography on both eyes to measure VD values (i.e., the experimental data) at Week 0 (i.e., Day 0). Experimenters assembled a glasses clip without an RF circuit 11 to the right glasses leg of each subject (i.e., regarded as control group), and assembled a glasses clip with an RF circuit 11 to the left glasses leg of each subject (i.e., regarded as experimental group). The appearances of the two types of glasses clips were completely the same. Therefore, each subject had both the control group and the experimental group, so that the effect caused by factors such as physiological conditions and eye use time among individuals during statistical analysis may be reduced.

At Week 2 (i.e., Day 14) and Week 4 (i.e., Day 28) after the glasses clips were assembled, all subjects returned for re-examination, and retinal microangiography on both eyes was performed again with the same instrument to measure the VD values (i.e., the experimental data) at Week 2 and Week 4. The re-examination date was limited to +/−2 days. If it exceeded this period, the experimental data would be excluded. After the VD value measurement at Week 4 was completed, the glasses clips were retrieved and stored according to their left and right positions, and were then brought back to the laboratory for testing whether the RF functions of the glasses clips retrieved from experimental group were normal or not. The experimental data of experimental group with the abnormal RF function would be excluded. After the experimental data sorting according to the exclusion conditions, the rest experimental data was used for statistical analysis of the following 5 items: (1) VD value changes; (2) trends of VD value changes over time; (3) effect of age on VD value changes; (4) effect of diseases on VD value changes; and (5) response rate analysis.

Since the left and right eyes of the same individual are not anatomically or functionally paired and exhibit differences, the experimental data of each eye should be regarded as an independent observation value. Therefore, the experimental data was statistically analyzed using unpaired t-test, and the statistical experimental data was expressed as the mean and the standard error of the mean (i.e., Mean±SEM). The trends of VD value changes over time and the response rate analysis were further statistically analyzed using repeated measures ANOVA to obtain an F value (statistical value).

In the figures, “*” indicates P<0.05, “**” indicates P<0.01, and “***” indicates P<0.001, all of which indicate statistically significant differences among multiple groups of data. “ns” indicates P>0.05, which indicates no statistically significant difference among multiple groups of data.

### D. Measurement and Analysis Method of VD Values

The retinal VD values of each subject is examined and calculated with an ophthalmic optical coherence tomography instrument (Avanti RTVue XR High Definition SD-OCT: Angio-OCT).

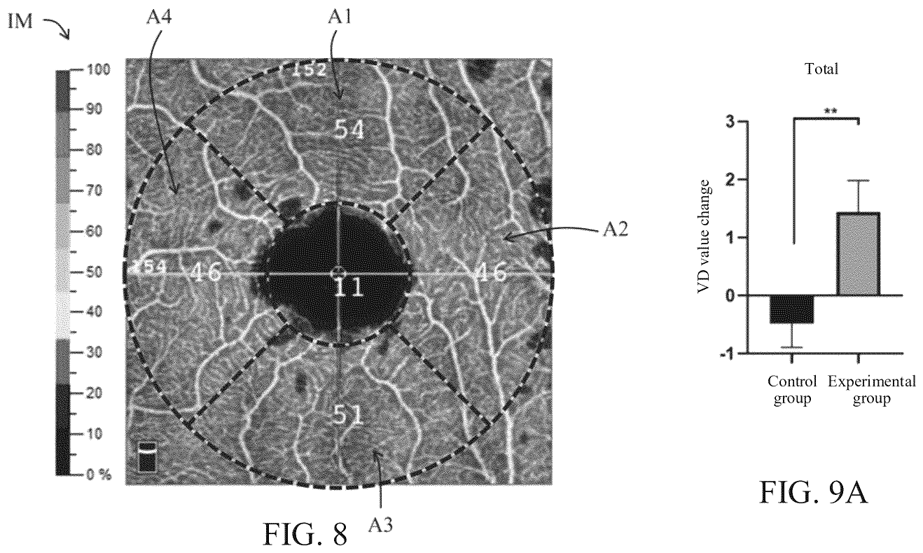

Through the optical coherence tomography angiography (OCTA) technology of this instrument, retinal angiography may be performed in a non-invasive manner. Then, the captured image IM is divided into four eye areas A1 to A4 (as shown in FIG. 8), i.e., superior, right, inferior, and left eye areas, and the VD of each eye area A1/A2/A3/A4 is quantified to obtain the corresponding VD value. Under normal circumstances, a total of 8 sets of experimental data corresponding to eye areas A1 to A4 may be acquired from both eyes of each subject.

In statistical analysis, by considering the dynamic change characteristics of vascular bed density and the future clinical application objects, besides total VD analysis, the experimental data is further divided into three groups based on the VD values: a low-density area (VD value ≤39), a medium-density area (40≤VD value ≤49), and a high-density area (VD value ≥50) for individual statistical analysis. This approach better reflects different physiological differences of the eye areas A1 to A4 and evaluates the effect of a non-thermal RF microwave EF on different capillary density areas. The low-density area (VD value ≤39) represents an eye area possibly having extensive focal ischemia, the medium-density area (40≤VD value ≤49) represents an eye area possibly having a small area of focal ischemia, and the high-density area (VD value ≥50) represents an eye area clearly having no focal ischemia.

### E. Experiment Result

39 subjects were recruited for this trial. Among them, 5 subjects failed to attend the scheduled tests, and 2 subjects experienced RF circuit 11 malfunction. Therefore, after excluding these 7 subjects, the test results of 32 subjects were finally included in the analysis. In addition, among the 32 subjects finally included, one subject had abnormal images of the right eye at Week 4, was suspected of having inflamed and congested vessels, and was referred to a physician for treatment. The images of the right eyes of two subjects were misaligned, resulting in the loss of data in one eye area. This might be caused by the gaze deviation during the examination. That is, the right eyes of these 3 subjects met the exclusion criteria, so the experimental data of their right eyes were excluded, while the experimental data of their left eyes were still included in the analysis. The amount of experimental data finally included in the analysis was shown in Table 2.

TABLE 2

| | Quantity of eye areas A1 to A4 | | | |
|---|---|---|---|---|
| | | VD | 40 ≤ VD | VD |
| | Total | value ≤ 39 | value ≤ 49 | value ≥ 50 |
| Control group | 116 | 21 | 67 | 28 |
| | | 18% | 58% | 24% |
| Experimental group | 128 | 49 | 60 | 19 |
| | | 38% | 47% | 15% |

### (1) VD Value Change

The VD value change of each group was calculated according to the following Formula 1.

$$VD value change = VD value at Week 4 - VD value at Week 0 \qquad Formula 1$$

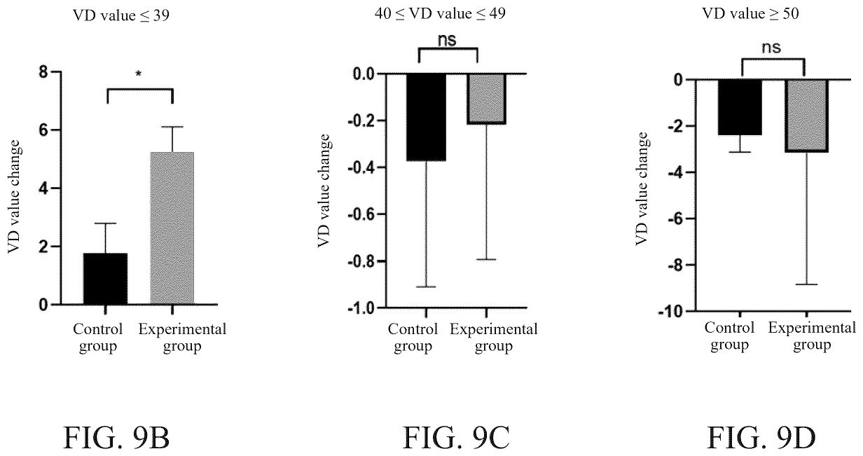

Referring to FIG. 9A, from the total, the mean and the standard deviation of the VD value change of experimental group and control group were 1.919±0.6928, and there was a significant difference (P=0.006). Through further observation on different capillary density areas, referring to FIG. 9B to 9D, it could be found that the significant VD value changes at Week 0 and Week 4 occurred in the low-density area with VD values ≤39. From the low-density area (VD value ≤39), it could be seen that the mean and standard deviation of the VD value changes between experimental group and control group were 3.50±1.467, and there was a significant difference between the two groups of data (i.e., P=0.0197), as shown in FIG. 9B. From the medium-density area (40≤VD value ≤49), it could be seen that the mean and standard deviation of the VD value changes in experimental group and control group were 0.1565±0.7862, and there was no significant difference between the two groups of data (i.e., P=0.8426), as shown in FIG. 9C. From the high-density area (VD value ≥50), it could be seen that the mean and standard deviation of the VD value changes between experimental group and control group were 0.1565±0.7862, and there was no significant difference between the two groups of data (i.e., P=0.8426), as shown in FIG. 9D.

Therefore, it could be known that RF microwave EF can increase the VD value, that is, the VD may be increased through the non-thermal effect induced by the RF microwave EF. Moreover, the effect of the RF microwave EF in high-VD areas is not obvious, and apparently, the initial VD value may affect the effect of RF microwave EF. In other words, the RF microwave EF with a non-thermal effect can increase the VD in low-VD areas without abnormally increasing the VD in normal areas.

### (2) Trends of VD Value Changes Over Time

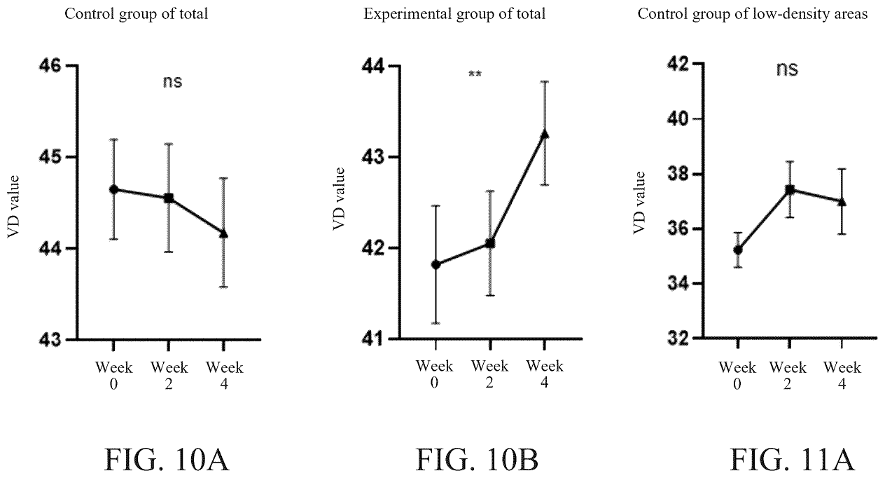

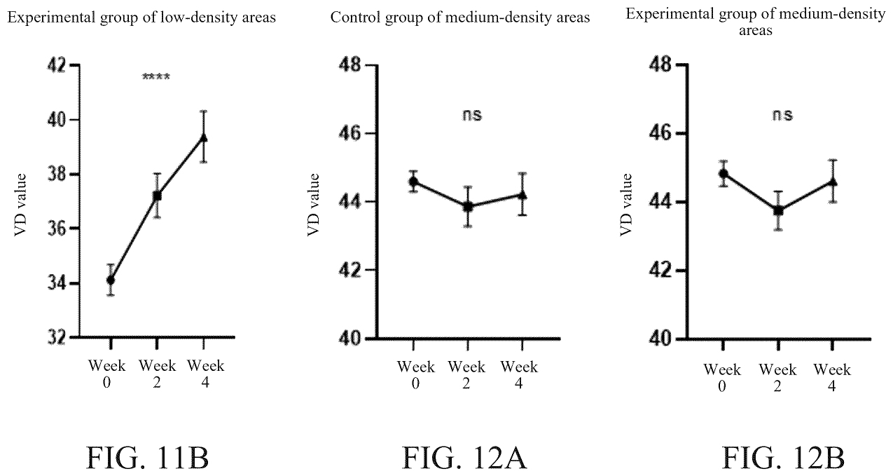

From the total, the VD values of control group did not change significantly from Week 0 to Week 4, as shown in FIG. 10A. However, the VD values of experimental group from Week 0 to Week 4 showed an upward trend and were significant, as shown in FIG. 10B. Through further observation on different capillary density areas, it could be found that the VD values of control group in the low-density areas (VD value ≤39) also did not significantly change from Week 0 to Week 4 and kept relatively stable, as shown in FIG. 11A. Relatively, the VD values of experimental group in low-density areas (VD value ≤39) from Week 0 to Week 4 showed an upward trend and were significant, as shown in FIG. 11B. In low-density areas (VD value ≤39), referring to FIG. 11B, the mean and standard deviation of the VD values of experimental group were increased from 34.12±0.557 at Week 0 to 37.22±0.808 at Week 2, and was increased to 39.39±0.932 at Week 4.

In medium-density areas (40≤VD value ≤49), referring to FIG. 12A to FIG. 12B, the F value of the VD value of control group from Week 0 to Week 4 was 1.082, and the P value was 0.3405. The F value of the VD value of experimental group from Week 0 to Week 4 was 2.576, and the P value was 0.0869. That is, from Week 0 to Week 4, there was no significant difference between experimental group and control group of the medium-density areas. That is, the VD value change curve was basically consistent.

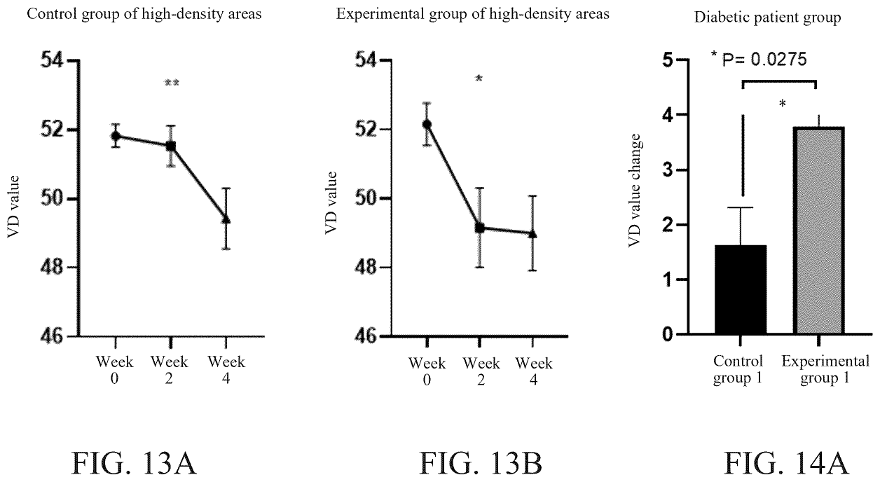

In high-density areas (VD value ≥50), referring to FIG. 13A to FIG. 13B, the F value of the VD value of control group from Week 0 to Week 4 was 8.955, and the P value was 0.0019. The F value of the VD value of experimental group from Week 0 to Week 4 was 2.576, and the P value was 0.0869. From FIG. 13A to FIG. 13B, it could be found that the mean and standard deviation of the VD value of experimental group in high-density areas at Week 0 and Week 2 were −2.714±1.246. In comparison to that of control group, the P value was 0.0372. That is, experimental group of the high-density area faster reflected the VD value decrease trend, but the final result was the same as that of control group (i.e., the VD values at Week 4 were similar).

It could be known that the RF microwave EF with a non-thermal effect can increase the VD in low-VD areas without abnormally increasing the VD in normal areas, and also can promote the VD in an area with a little higher VD to recover the VD of the normal area.

### (3) Effect of Diseases on VD Value Changes

In aspects of studying the effect of diseases, the experimental data of 6 diabetic patients among the 32 eligible subjects was observed, moreover, the experimental data of non-diabetic patients and diabetic patients in low-density areas (VD value ≤39) were also compared.

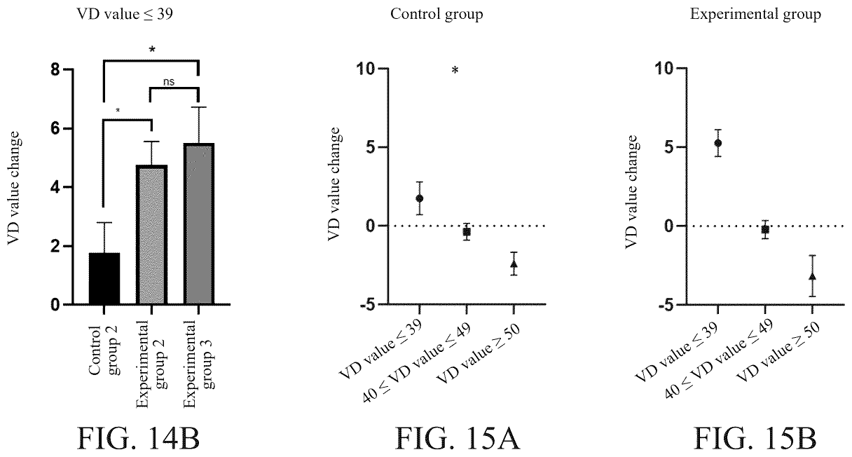

Referring to FIG. 14A, in a diabetic patient group, the mean and standard deviation of the VD value changes of control group 1 (n=16) were 1.625±0.6884, and the mean and standard deviation of the VD value changes of experimental group 1 (n=24) were 3.792±0.6197.

Referring to FIG. 14B, in low-density areas (VD value ≤39), the mean and standard deviation of the VD value changes of control group 2 (n=21) of a non-diabetic patient group were 1.762±1.039, the mean and standard deviation of the VD value changes of experimental group 2 (n=16) of a diabetic patient group were 4.75±0.8036, and the mean and standard deviation of the VD value changes of experimental group 3 (n=33) of a non-diabetic patient group were 5.515±1.206. In the above, n is the quantity of samples, i.e., the quantity of eye areas A1 to A4.

In the diabetic patient group, the mean and standard deviation of the difference value of the VD value changes of experimental group 1 and the VD value changes of control group 1 were 1.925±1.004, and the P value was 0.0275. In low-density areas (VD value ≤39), the mean and standard deviation of the difference value of the VD value changes of experimental group 2 and the VD value changes of control group 2 were 2.988±1.384, and the P value was 0.0378. In addition, in low-density areas (VD value ≤39), the mean and standard deviation of the difference value of the VD value changes of experimental group 2 and the VD value changes of experimental group 3 were 0.7652±1.827, and the P value was 0.6773.

Therefore, the diabetes does not seem to limit the effect of the RF microwave EF with a non-thermal effect.

### (4) Response Rate Analysis

In the response rate analysis, statistical analysis was performed on whether the VD values of the subjects increased, kept unchanged or decreased, and the results of the statistical analysis are shown in Table 3, FIG. 15A and FIG. 15B.

TABLE 3

| | Quantity of | VD value | VD value kept | VD value |
|---|---|---|---|---|
| Group | samples | increased | unchanged | decreased |
| VD | 49 | 40 | 4 | 5 |
| value ≤ 39 | | 81.6% | 8.2% | 10.2% |
| 40 ≤ VD | 60 | 28 | 9 | 23 |
| value ≤ 49 | | 46.7% | 15.0% | 38.3% |
| VD | 19 | 1 | 6 | 12 |
| value ≥ 50 | | 5.3% | 31.6% | 63.2% |

*Data of each group was expressed by the quantity of samples (top) and the proportion in the group to which it belonged (bottom).

From the statistical analysis results, it could be found that referring to Table 3, in the low-density areas with a relatively low initial VD value (VD value ≤39), the RF microwave EF may significantly increase the VD value. Among them, the VD values of 81.6% individuals increased, the VD values of 10.2% individuals decreased, and the VD values of 8.2% individuals kept unchanged. In medium-density areas (40≤VD value ≤49), the VD values of 46.7% individuals increased, the VD values of 38.3% individuals decreased, and the VD values of 15.0% individuals kept unchanged. In high-density areas (VD value ≥50), the VD values of only 5.3% individuals increased, the VD values of 63.2% individuals decreased, and the VD values of 31.6% individuals kept unchanged.

Referring to FIG. 15A and FIG. 15B, in control group, the means and standard deviations of the VD value changes of three groups (VD value ≤39, 40≤VD value ≤49, and VD value ≥50) were sequentially 1.76±1.04, −0.37±0.54, and −2.39±0.73. In experimental group, the means and standard deviations of the VD value changes of three groups (VD value ≤39, 40≤VD value ≤49, and VD value ≥50) were sequentially 5.27±0.85, −0.22±0.58, and −3.16±1.30. It was further discovered that the differences of the VD value changes of control group were small among different capillary densities (F=5.548, and P=0.005), as shown in FIG. 15A. Relatively, there were significant differences of the VD value changes of experimental group among different capillary densities (F=23.25, and P<0.0001), as shown in FIG. 15B.

From the above trial, it could be known that the RF microwave EF with a non-thermal effect achieves the acting range being selective on the initial VD value, and can increase the lower VD and decrease the higher VD to achieve the bidirectional regulation on the retinal VD. Particularly, through the non-thermal effect of RF microwave EF, the VD value of the retina may be effectively increased, even for diabetic patients, and it will not cause abnormal increase of VD in normal areas. Particularly, in low-VD eye areas (for example, VD value ≤39), the VD may be significantly improved through the effect of the RF microwave EF with a non-thermal effect. That is, the non-thermal effect of RF microwave EF can induce a protective mechanism on vascular endothelial cells and promote the vascular network to cooperate more actively with physiological regulation and control, thereby increasing the VD value in the low-VD eye areas. In addition, for subjects with long exposure time to the RF microwave EF, the retinal VD may also be significantly improved through the effect of the RF microwave EF with a non-thermal effect.

Based on the above, the method for improving retinal vascular circulation using RF microwaves EF, the RF emitting circuit 10 using the same and the use thereof provided by any one embodiment may be used for applying a non-thermal effect to an eye 20 by emitting the RF microwaves with a frequency between 30 MHz and 3 GHz, thereby substantially imperceptibly regulating retinal VD and improving the vascular circulation. Specifically, in some embodiments, through the method for improving retinal vascular circulation using RF microwaves EF, the RF emitting circuit 10 using the same and the use thereof, the too low VD may be increased, and the too high VD may be decreased, so that the blood flow distribution of the retinal capillary may be optimized to improve vascular circulation.

What is claimed is:

1. A method for regulating retinal vessel density (VD) using radio frequency (RF) microwaves, comprising:
positioning an RF emitting circuit within a range of 3 cm to 10 cm away from an eye;
driving the RF emitting circuit to emit an RF microwave with a frequency between 30 MHz and 3 GHz; and
applying a non-thermal effect to a retinal area of the eye by emitting the RF microwave.

2. The method according to claim 1, wherein the RF emitting circuit is a passive RF circuit.

3. The method according to claim 1, wherein the RF emitting circuit is an active RF circuit.

4. The method according to claim 1, wherein the RF emitting circuit is a glasses clip or a pair of glasses.

5. A method for regulating retinal VD using an RF emitting circuit, wherein the RF emitting circuit is configured to apply an RF microwave with a frequency between 30 MHz and 3 GHz in a non-contact manner.

6. An RF emitting circuit for regulating retinal VD, comprising:
an emitting module; and
a control module, connected to the emitting module and configured to control the emitting module to generate an RF microwave with a frequency between 30 MHz and 3 GHz.

7. The RF emitting circuit according to claim 6, wherein the control module is a passive chip.

8. The RF emitting circuit according to claim 6, further comprising:
a power supply module, connected to the control module and configured to supply power to the control module.

9. The RF emitting circuit according to claim 6, further comprising: a pair of glasses, wherein the emitting module and the control module are disposed on the pair of glasses.
